(19) United States
(12) Patent Application Publication
(10) Pub. No.: US 2016/0015472 A1
(43) Pub. Date: Jan. 21, 2016

(54) Methods for Detecting Materials in a Body Cavity

(71) Applicants: Aaron Matthew NOBLE, Newnan, GA (US); John Patrick TIERNAN, Newnan, GA (US); Lawrence Scott ARONSON, Peachtree City, GA (US)

(72) Inventors: John Patrick Tiernan, Newnan, GA (US); Lawrence Scott Aronson, Peachtree City, GA (US)

(21) Appl. No.: 14/776,768

(22) PCT Filed: Mar. 14, 2014
(86) PCT No.: PCT/US2014/029113

§ 371 Date: Sep. 15, 2015

Related U.S. Application Data
(60) Provisional application No. 61/794,888, filed on Mar. 15, 2013.

(51) Int. Cl. A61B 19/00; A61B 17/28; A61B 17/06; A61B 17/3211; A61F 13/44; A61F 13/36

(52) U.S. Cl. CPC A61B 19/5244; A61F 13/44; A61F 13/36; A61B 17/06166; A61B 17/3211; A61B 17/28; A61B 2019/547; A61B 2019/5495; A61F 2013/530233

(57) ABSTRACT

The present disclosure relates to a medical material or surgical sponge including an integral construction having a plurality of micro-particle taggants that are UV or radio wave detectable. The sponge includes a fibrous, nonwoven fabric containing entangled fibers arranged in an interconnecting patterned relationship in a plane of the fabric, and at least one UV or radio wave detectable element positioned interiorly of the fibrous nonwoven fabric in the plane. The fibers of the nonwoven fabric may be inter twined about the micro-particle taggants.

### CROSS REFERENCES TO RELATED APPLICATIONS

This application claims the benefit of U.S. Provisional Patent Application Ser. No. 61/794,888, entitled: “Methods for Detecting Materials in a Body Cavity,” filed Mar. 15, 2013, the content of which is incorporated herein by reference in its entirety.

### FIELD

This disclosure generally is directed to medical materials and surgical materials constructed of fibrous fabric materials. More particularly, this disclosure is directed to a surgical sponge and other devices having a detectable, and identifiable measure to combat inadvertently leaving it in a body cavity following a surgical procedure.

### SUMMARY

In one aspect, this disclosure can be summarized as medical materials and surgical materials (e.g., a surgical sponge) comprising a fabric having one or more micro-particle taggant detectable elements as an integral part of the fibrous fabric construction obtained by integrating a plurality of micro-particle taggants with multiple layers and having one or both of a fluorescing agent and a magnetic charge for initial detection of the presence of the surgical sponge containing the taggant(s) (e.g., a micro-particle for detection by radio waves or radiation).

In another aspect, a medical material is disclosed. The medical material includes a fibrous, nonwoven fabric comprised of entangled fibers arranged in an interconnecting patterned relationship in a plane of the fabric, and at least one micro-particle taggant including at least one of a ultraviolet detectable element and a radio wave detectable element disposed in an interior of the fabric, wherein the fibers are intertwined about the micro-particle taggant.

In another aspect, a medical sponge is disclosed. The medical sponge includes a first outer fabric layer, a second outer fabric layer, and an intermediate layer disposed between the first and second outer layers, wherein the intermediate layer including a micro-particle taggant including at least one of a ultraviolet detectable element and a radio wave detectable element embedded in the intermediate layer.

In yet another aspect, a method of making a medical material is disclosed. The method includes blending a plurality of micro-particle taggants having multiple colored layers with a plastic resin, and molding or spinning a fiber material during the blending such that the micro-particle taggants are embedded in the fibers.

### BRIEF DESCRIPTION OF THE DRAWINGS

Embodiments of devices, systems, and/or methods are illustrated in the figures of the accompanying drawings which are meant to be exemplary and not limiting, in which like references are intended to refer to like or corresponding parts, and in which:

FIG. 1 illustrates a surgical sponge according to an aspect of the present disclosure;

FIG. 2 illustrates micro-particle taggants integrated into a material according to an aspect of the present disclosure;

FIG. 3 illustrates a multi-layer article having micro-particle taggants integrated into an intermediate layer according an aspect of the present disclosure;

FIG. 4 illustrates a multi-layer article having micro-particle taggants integrated into an outer layer according an aspect of the present disclosure;

FIG. 5 illustrates an ultraviolet light emitting device;

FIG. 6 illustrates a radio wave emitting device; and

FIG. 7 illustrates a block flow diagram of a method according to an aspect of the present disclosure.

### DETAILED DESCRIPTION

Detailed embodiments of devices, systems, and methods relating to detection materials in a body cavity are disclosed herein, however, it is to be understood that the disclosed embodiments are merely exemplary. Therefore, specific functional details disclosed herein are not to be interpreted as limiting, but merely as a basis for the claims and as a representative basis for teaching one skilled in the art to variously employ the present disclosure.

In an aspect, a medical material or surgical sponge is disclosed. The medical material or surgical sponge includes an integral construction having a plurality of micro-particle taggants that are ultraviolet (UV) or radio wave detectable. The medical material or surgical sponge may be constructed of a fibrous, nonwoven fabric containing entangled fibers arranged in an interconnecting patterned relationship in the plane of the fabric, and at least one UV or radio wave detectable element positioned in an interior of the fibrous nonwoven fabric. The fibers of the nonwoven fabric are intertwined about the micro-particle taggants.

For example, the UV or radio wave detectable element may be a yarn and the fibers of the nonwoven fabric are intertwined with the fibers of the yarn. The yarn may be a multi-ply, twisted yarn of viscose fibers containing UV or radio wave detectable material. The UV or radio wave detectable element may be a monofilament comprising a thermoplastic polymeric material containing color fluorescing micro-particles. For example, the polymeric material may be one or more of polyisobutylene, polyvinyl chloride, and copolymers of vinyl acetate and vinyl chloride.

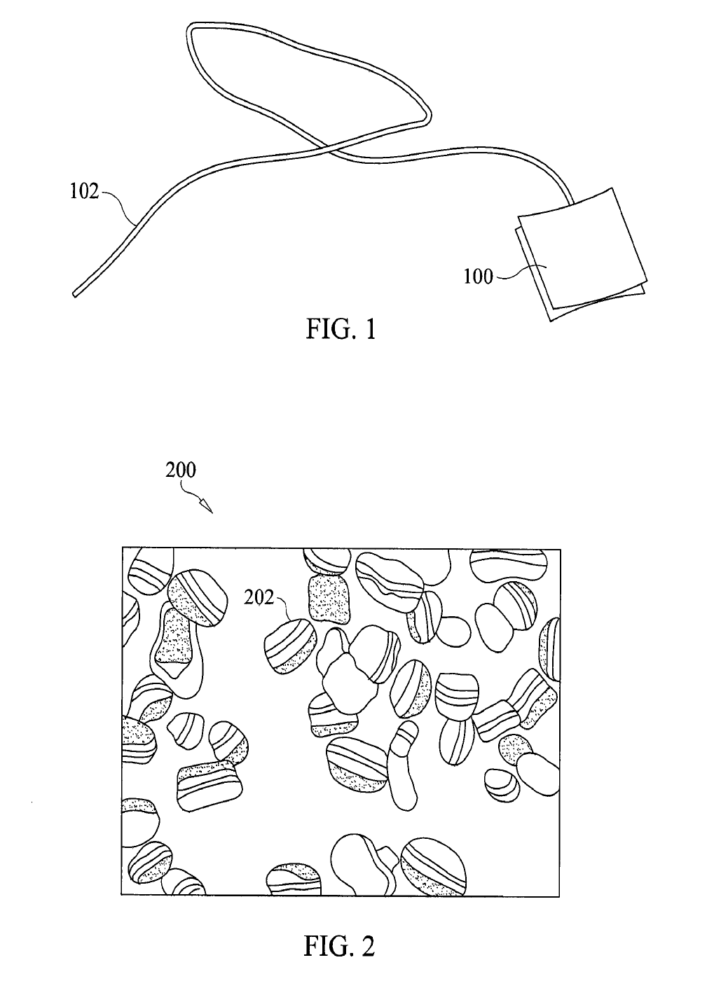

In an aspect, a medical/surgical material, for example, a surgical sponge is disclosed having a fabric including one or more micro-particle taggant detectable elements integrated into the fibrous fabric construction. Referring to FIG. 1, in an illustrative embodiment, a surgical sponge 100 includes a material or layer having one or more micro-particle taggants integrated and adhered to the construction. The surgical sponge may have a porosity that is macro-porous (i.e. larger than 50 nanometers). The surgical sponge may also include a string 102, or other tab, coupled to an end of the sponge 100 for gripping with a hand or surgical device, such as forceps. One or more micro-particle taggants may also or alternatively be integrated into the string 102.

In an illustrative embodiment, a material 200 including one or more multi-layer colored fluorescing micro-particles and/or radio wave detectible material is described with reference to FIG. 2. As illustrated in FIG. 2, the material 200 includes one or more multi-layer colored fluorescing micro-particles 202 integrated and adhered to the construction. The micro-particles 202 may include one or more layers. As illustrate din FIG. 2, the micro-particles 202 include 4 layers. Each layer may be a different color and to convey different information when the micro-particles 202 are detected. For example, the micro-particles may provide a code for identifying a product, device, article, or other material.

For example, U.S. Patent Application Publication No: US 2002/0129523 discloses the use of multi-layer colored micro-particles adhered to an object, the disclosure of which is incorporated by reference in its entirety. As described in U.S. Patent Application Publication No: US 2002/0129523, the micro-particles may include two or more distinguishable marker layers corresponding to a predetermined numeric code. The marker layers may also each include a different color or color enhancer. A plurality of micro-particles may comprise a plurality of micro-particle sets, wherein each micro-particle set is characterized by a specific marker layer combination different from each other micro-particle set and the combination of micro-particle sets employed collectively forms the numeric code. The micro-particle sets may also include at least one datum marker layer, which functions to identify an orientation of the value marker layers coded and is also coded to include place information. Further, the micro-particle taggants may be formulated with a binder, such as an adhesive or coating, capable of coupling the taggants to an object or material.

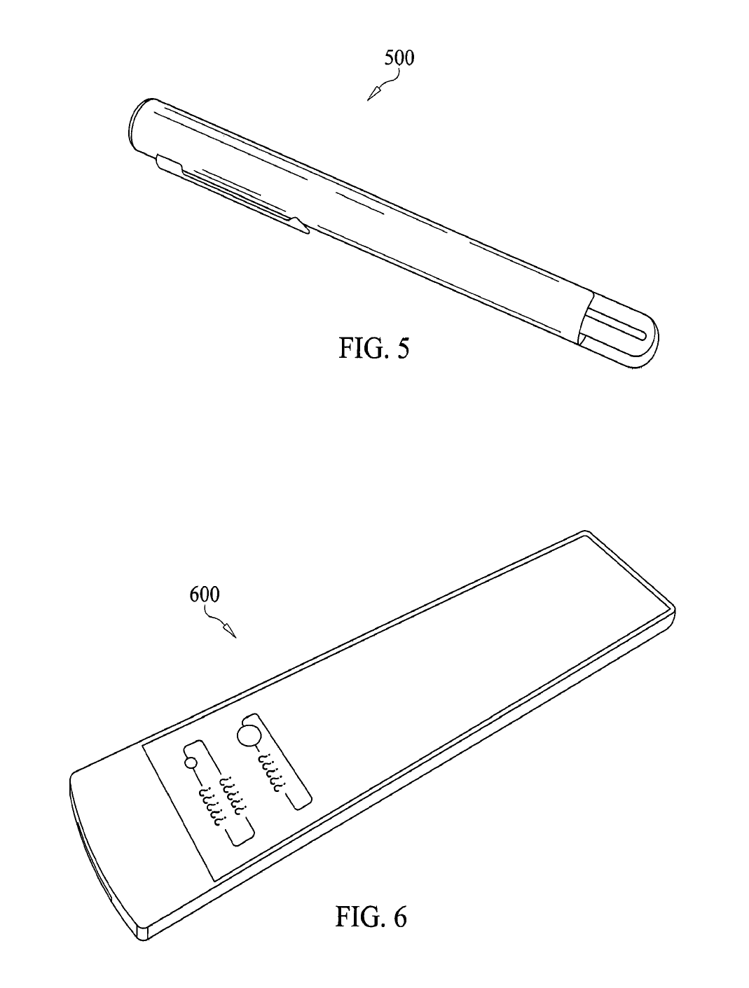

In an aspect, the micro-particles 202 are detectible in the presence of non-visible UV light and/or radio waves. For example, the micro-particles 202 may illuminate in the presence of UV light, for example using a UV light emitting device, such as UV emitting device 500 illustrated in FIG. 5. Similarly, the micro-particles 202 may be detected by radio waves, for example using a radio wave emitting device, such as the radio wave emitting device 600 illustrated in FIG. 6 that produces an audible sound when it detects the micro-particles.

The UV emitting device 500 and the radio wave emitting device 600 can be hand held battery operated, and as small as a laser pointer, as illustrated in FIGS. 5 and 6. Surgical teams may use these devices to inspect the surgical cavity for the presence of a lost surgical article, such as a sponge. For example, since UV light is only visible when intersecting the surgical sponge containing the micro-particles 202, the surrounding areas remain non-illuminated providing sharp contrast to the surrounding areas and making the surgical sponge discernable even in dark or shadowed areas. This use of color fluorescing micro-particles 202 integrated into the surgical sponge materials provides an improvement in detection.

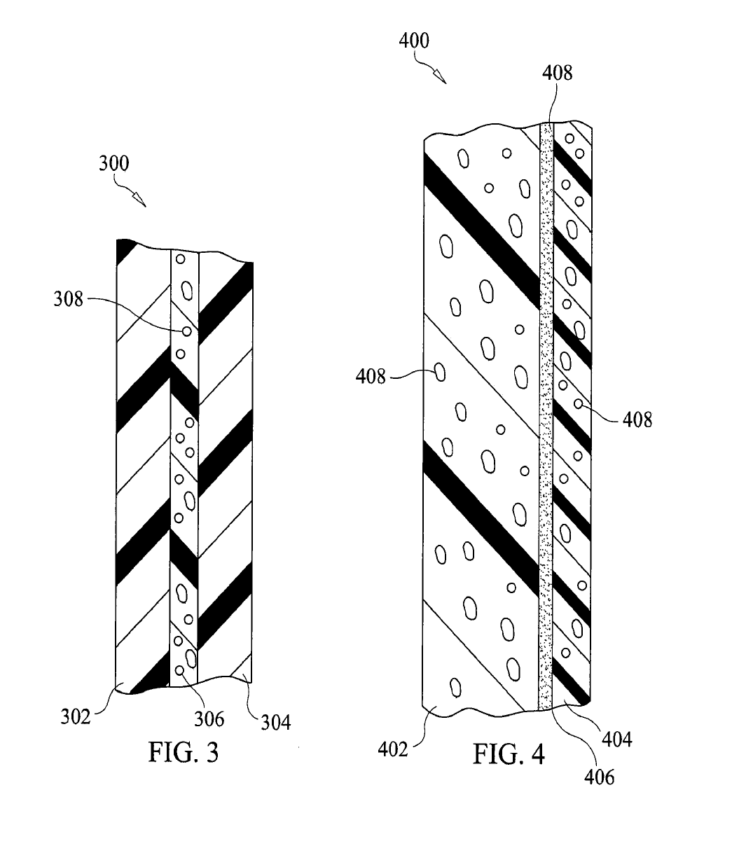

The micro-particles may be integrated into one or more intermediate layers and/or outer layers of a sponge or other material. Referring to FIG. 3, in an aspect, a sponge 300 includes a first outer layer 302, a second outer layer 304, and at least one intermediate layer 306 disposed between the outer layers 302 and 304. As illustrated, micro-particles 308, which may be the same as micro-particles 202, are integrated into the intermediate layer 306.

In another aspect, referring to FIG. 4, a sponge 400 may include a first outer layer 402, a second outer layer 404, and at least one intermediate layer 406 disposed between the outer layers 402 and 404. Micro-particles 408, which may be the same as micro-particles 202, may be integrated into one or more of the layers 402-406. As illustrated in FIG. 4, the micro-particles 408 are integrated into each of the layers 402-406; however it should be appreciated that any one of the layers 402-406 may be constructed without the micro-particles 408.

In an aspect, integration of the micro-particle taggant into a material, such as a surgical sponge includes blending the micro-particle taggant in a resin prior to or during molding or extruding or spinning of a surgical sponge constructed of fibrous materials. In a preferred embodiment, the micro-particle taggant is a plurality of micro-particle taggants with multiple layers and having one or both of a fluorescing agent and a magnetic charge for initial detection of the presence of the surgical sponge containing the micro-particle taggant.

In an aspect, the micro-particles have a size ranging from about 20 to about 600 microns, and more particularly, the micro-particles range in size from about 44 to about 75 microns. The micro-particle may be present in a concentration from about 0.8 to about 47 particles per square centimeter (i.e. about 5 to about 300 particles per square inch).

The micro-particle taggants may also be provided with other identifiable features. For example, the micro-particle taggants may be provided with a fluorescing agent of selected colors. The fluorescing agent may be any particularly discernable material under UV light, and is provided on the micro-particle taggants as one or more faces or as one of the layers. Micro-particle taggants are commercially available, such as from Microtrace, LLC of Minneapolis, Minn.

This allows a surgical site to be initially inspected for the presence of a lost surgical sponge or other article containing the micro-particle taggants and, once the missing surgical sponge is found, the package can thereafter be further inspected to identify the code designed into the micro-particle taggants to provide a high level of tractability in the event the surgical sponge is inadvertently torn from the attached string. In another example, the micro-particle taggants may be magnetized to a particular attractive strength so as to provide a unique magnetic signature for further detecting and tracing the surgical sponge or other article. In the event that an obstruction blocks the UV light, the use of a radio wave emitting device that can detect the surgical sponge by generating an auditable tone, such as the device 600 illustrated in FIG. 6.

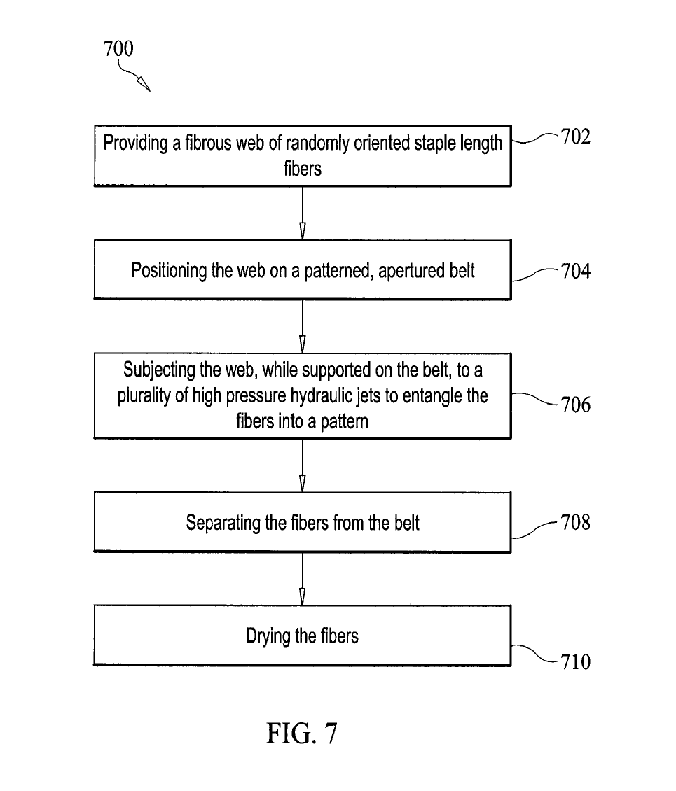

In an aspect, the surgical sponges are fabricated of fabrics integrated with micro-particle taggants manufactured according to conventional hydraulic entanglement methods. Referring to FIG. 7, a method 100 includes providing a fibrous web of randomly oriented staple length fibers, illustrated as block 702; positioning the web on a patterned, apertured belt, illustrated as block 704; and subjecting the web while supported on the belt to a plurality of high pressure hydraulic jets to entangle the fibers into a pattern conforming to that of the supporting belt, illustrated as block 706. The entangled fibers are thereupon separated from the belt, illustrated as block 708, and dried on hot drums, illustrated as block 710, to produce a patterned nonwoven fabric. This method of manufacturing is described in detail in U.S. Pat. Nos. 3,068,547; 3,129,466; 3,485,706; 3,494,821; and 3,681,184, the disclosures of which as incorporated by reference in their entirety.

The nonwoven fabric may comprise any suitable combination of natural and/or synthetic textile materials including cotton, rayon, nylon, cellulosics, acrylics, polyamides, polyesters, polyolefin, and blends thereof. One exemplary fiber composition is a blend of about 70% by weight rayon (1.5 denier, approximately 3 cm staple length) and about 30% by weight polyester (1.5 denier, approximately 3 cm staple length). The staple fibers are blended and converted to a fibrous web on conventional textile processing equipment such as a Rando-Webber which produces a web having random fiber orientation.

In the manufacture of the nonwoven fabrics, two fibrous webs produced from the staple fiber blend are laid one upon one another on a moving belt. At the same time, one or more strands integrated with UV and/or radio wave detectable micro-particle taggants are positioned between the two webs. The composite material is carried by the belt through the hydraulic entanglement process whereupon the individual webs are unified to form a single thickness of nonwoven fabric with the UV and or radio wave detectable element positioned interiorly thereof, for example, as described and illustrated in connection with FIG. 3. The composite nonwoven fabric is thereupon removed from the belt, dried over heated drums, and collected on a roll.

In an example, the unified, nonwoven fabric preferably has a total dry weight of from about 1.0 to about 3.0 ounces per square yard (about 30 to about 100 g/m²), with the lighter weights limited by the process ability of the fibrous webs and the heavier weights limited by the desired utility and construction of the sponge, although higher weights may be preferred for some product applications such as laparotomy pads.

The UV and/or radio wave detectable material may be blended into any continuous filament, yarn or ribbon of sufficient density to provide an acceptable degree of contrast when exposed to UV light, such as the UV emitting device 500 illustrated in FIG. 5, or a radio wave emitting device, such as the radio wave emitting device 600 illustrated in FIG. 6. A suitable monofilament is polyvinylchloride, about 0.635 mm (about 0.025 in.) in diameter. A suitable yarn is made from a viscose staple containing micro-particle taggants which is spun into a 60 tex singles yarn. Four, six or eight ply yarns may be made from the single yarns for incorporation into the nonwoven fabric.

In an embodiment, a continuous length of nonwoven fabric containing the UV and/or radio wave detectable taggants may be converted into multi-ply surgical sponges using conventional techniques.

In an embodiment, the fabrics may be constructed of any suitable fibrous material, and in a variety of patterns, all of which are well within the skill of the art. The fibrous material may, for example, be selected from the group consisting of cotton, rayon, cellulosics, acrylics, polyamides, polyesters, polyolefins, and blends thereof.

Porous plastics, such as plastics having a macro-porous porosity (i.e. larger than 50 nanometers) and other plastics, may also be made by a form of sintering. Sintering is the process of fusing discrete particles by heat, with or without pressure, to form a porous structure. The sintering process uses raw material in the form of discrete particles of a thermoplastic polymer having a plurality of micro-particle taggants that are UV and/or radio wave detectable blended in the particles prior to sintering. The sintered shape may be in a form suitable for use as a surgical sponge or other medical device/article. For example, a porous material may be made of sintered particles having an average particle size of about 40 to about 600 microns, made up of, but not limited to, polyethylene, polypropylene, and/or ultra-high molecular weight polyethylene (UHMW).

Although the devices, systems, and methods have been described and illustrated in connection with certain embodiments, many variations and modifications should be evident to those skilled in the art and may be made without departing from the spirit and scope of the disclosure. For example, while many of the examples are directed to surgical sponges, it is clear that the techniques herein and fibers herein can be incorporated into other medical materials and articles, such as gauze sutures, scalpels, forceps, packaging, pouches, caps, and other materials. Further, the methods and techniques can be used with coating or impregnation into resins (i.e. for sutures, and instruments). The discourse is thus not to be limited to the precise details of methodology or construction set forth above as such variations and modification are intended to be included within the scope of the disclosure.

What is claimed is:

1. A medical material, comprising:
a fibrous, nonwoven fabric comprised of entangled fibers arranged in an interconnecting patterned relationship in a plane of the fabric; and
at least one micro-particle taggant including at least one of a ultraviolet detectable element and a radio wave detectable element disposed in an interior of the fabric, wherein the fibers are intertwined about the micro-particle taggant.

2. The medical material of claim 1, wherein the at least one of the ultraviolet detectable element or the radio wave detectable element is a yarn, and the fibers of the fabric are intertwined with the yarn.

3. The medical material of claim 2, wherein the yarn is a multi-ply, twisted yarn of viscose fibers containing the at least one of the ultraviolet detectable element or the radio wave detectable element.

4. The medical material of claim 1, wherein the at least one of the ultraviolet detectable element or the radio wave detectable element is a monofilament comprising a thermoplastic polymeric material containing color fluorescing micro-particles.

5. The medical material of claim 4, wherein the thermoplastic polymeric material is selected from the group consisting of: polyisobutylene, polyvinyl chloride, and copolymers of vinyl acetate and vinyl chloride.

6. The medical material of claim 1, wherein the fabric includes fibrous material selected from the group consisting of: cotton, rayon, cellulosics, acrylics, polyamides, polyesters, polyolefin, and blends thereof.

7. The medical material of claim 1, wherein the fabric includes a blend of about 70% by weight rayon and about 30% by weight polyester staple fibers.

8. A medical device, comprising:
a first outer fabric layer;
a second outer fabric layer; and
an intermediate layer disposed between the first and second outer layers, the intermediate layer including a micro-particle taggant including at least one of a ultraviolet detectable element and a radio wave detectable element embedded in the intermediate layer.

9. The medical device of claim 9, wherein the intermediate layer includes multiple intermediate layers and the micro-particle taggant is embedded in one of the multiple intermediate layers.

10. The medical device of claim 9, wherein the micro-particle taggant includes at least one of a fluorescing agent and a magnetic charge element to enable detection of a presence of the micro-particle taggant.

11. The medical device of claim 9, wherein the micro-particle taggant includes one or more colored layers adapted to provide a code for identifying the medical sponge.

12. The medical device of claim 9, wherein the micro-particle taggant is embedded in the intermediate layer by a molding or spinning process.

13. The medical device of claim 9, further comprising a string coupled to an end of the medical sponge

14. The medical device of claim 13, wherein the string has a fibros construction and includes second micro-particle taggants disposed in the string.

15. The medical device of claim 14, wherein the second micro-particle taggants include one or more colored layers.

16. The medical device of claim 14, wherein the second micro-particle taggants include one or more colored layers.

17. The medical device of claim 14, wherein the medical device is a sponge.

18. The medical device of claim 14, wherein the medical device is a gauze, a suture, a scalpel, a forcep, a pouche, or a cap.

19. A method of making a medical material, comprising:
blending a plurality of micro-particle taggants having multiple colored layers with a plastic resin; and
molding or spinning a fiber material during the blending such that the micro-particle taggants are embedded in the fibers.

20. The method of claim 19, wherein the blending includes blending the micro-particle taggants having a size ranging from about 20 microns to about 600 microns with the plastic resin.

21. The method of claim 19, wherein the blending includes blending the micro-particle taggants having a size ranging from about 44 microns to about 75 microns with the plastic resin.

22. The method of claim 19, wherein the blending includes blending the micro-particle taggants with the plastic resin, wherein a concentration of the micro-particle taggants is about 0.8 particles per square centimeter to about 47 particles per square centimeter.

23. The method of claim 19, wherein the blending includes blending the micro-particle taggants including at least one of a ultraviolet detectable element and a radio wave detectable element with the plastic resin
